(19) United States
(12) Patent Application Publication
(10) Pub. No.: US 2007/0233133 A1
(43) Pub. Date: Oct. 4, 2007

(54) Bone-Evacuating and Valve-Exiting Resector and Method of Using Same

(76) Inventors: Taylor S. Cohen, Philadelphia, PA (US); Ralph G. Dacey JR., St. Louis, MO (US); Aaron Ferber, St. Charles, MO (US); Guy M. Genin, St. Louis, MO (US); Daniel Jansen, St. Charles, MO (US); Eric C. Leuthardt, St. Louis, MO (US); Adam C. Nathan, Fort Meyers, FL (US); Arash Sabet, Ballwin, MO (US)

Correspondence Address: THOMPSON COBURN LLP;ATTN: RICHARD E. HAFERKAMP, ONE U.S. BANK PLAZA, SAINT LOUIS, MO, 63101, US

(21) Appl. No.: 11/678,435

(22) Filed: Feb. 23, 2007

Related U.S. Application Data
(60) Provisional application No. 60/779,400, filed on Feb. 24, 2006.

(51) Int. Cl. A61B 17/00

(52) U.S. Cl. 606/83

(57) ABSTRACT

A pneumatically powered bone resector has a moveable cutting arm and a stationary foot against which the cutting arm homes to cut bone positioned therebetween, the cutting arm being hollow for receiving resected bone with a mechanical/pneumatic linkage connected to the cutting arm for driving the cutting arm and blowing the resected bone through the cutting arm and into a receiving bin, an electronically controlled pneumatic power supply for powering the linkage upon operation of a trigger, the trigger being operated by a surgeon.

### CROSS-REFERENCE AND PRIORITY CLAIM TO RELATED APPLICATIONS

This application claims priority to U.S. provisional patent application Ser. No. 60/779,400, entitled “Bone-Evacuating and Valve-Exiting Resector and Method of Using Same”, filed Feb. 24, 2006.

### BACKGROUND AND SUMMARY OF THE INVENTION

Surgical tools for resecting bone are well known in the prior art. These tools are used to reach inside an incision and resect or “chip” away the bone from a patient for any one of several reasons. This process generally takes place as the patient is under some form of anesthesia which dictates that the speed of the operation be given some urgency. Furthermore, it is also the case that prior art devices require a process of continual insertion and removal of the bone resector which is not only a time consuming and cumbersome process but also provides opportunity for infection due to the possible introduction of foreign materials into the incision. For these reasons the prior art devices have been used but are not in keeping with the modernization of many surgical techniques which take these risks into account.

One example of a bone resector presently in use is the Kerrison™ Rongeur. It allows a surgeon to remove sections of bone or tissue manually. The surgeon places the stop of the Kerrison rongeur (far left of FIG. 1) beneath the section of bone to be resected and squeezes the handle (see FIG. 1). This causes the cutting arm to rise up and, if sufficient force is applied, penetrate the bone to the depth of the curved end. The surgeon then removes the Kerrison™ Rongeur from the surgical entry, hands the cutting end of the tool to a nurse, and instructs the nurse to either keep or discard the resected bone or tissue. The nurse removes the bone or tissue fragment from the Kerrison rongeur, typically with her gloved fingers, and informs the surgeon when this task has been completed while handing the tool back to the surgeon for the next resection. The surgeon then reinserts the Kerrison™ Rongeur in to the surgical entry and the process repeats, sometimes dozens of times, until the surgeon finishes the bone resection.

To eliminate the above noted shortcomings of the prior art device and its method of use, the inventors have succeeded in developing a pneumatically powered bone resector and collection device which represents a significant improvement over the prior art device. The new invention may remain in the surgical opening while multiple resections are made, power the cutting operation so that the surgeon need only place the tool and not be required to squeeze or exert hand pressure to achieve the cutting, and automatically clear the tool for the next resection by blowing the resected bone tissue into a collection container which may automatically segregate the tissue specimens into one of a plurality of collection bins. The surgeon may then finish the resection procedure swiftly upon one insertion and then removal to minimize the risk of infection, diminish the time required to completion to minimize surgical risk to the patient, and relieve the surgeon from any tiring effects on his hand which ensure a safer and more accurate procedure.

The present invention is elegantly simple in design, adapted into a similar profile as that of the prior art device, both being substantially in the form of a hand held pistol for ease and accuracy of use, and will thus be readily adopted and accepted by a surgeon in his practice with little re-learning or unfamiliarity to overcome. In essence, and as explained in greater detail below, the present invention comprises a hand held device containing a trigger actuator, an air cylinder, a mechanical linkage powered by the air cylinder to drive the moveable cutting arm into the stationary foot to resect the bone therebetween, a collection box and two collection bins into which the resected bone tissue is blown, and five air lines—two of which drive the air cylinder, another air line which clears the cutting arm by blowing the bone tissue through its hollow length, and a last pair of air lines which blow the bone tissue specimen from a common sorting bin into one of a plurality of designated collection bins. Each sorting bin may contain a mechanism such as a one way valve to prevent bone chips from exiting the sorting bin, and indicators such as light emitting diodes or audio signal devices to indicate the extent to which the sorting bin's volume is filled, and thus when the bin requires emptying or replacement. The air lines are controlled by an electronic control so as to be timed with respect to each other and operate in proper sequence. Also, on the outside of the hand held tool are a toggle switch allowing the surgeon to designate the collection bin to receive the resected bone specimens and a trigger switch for the surgeon's use in making a resection once the tool is positioned as desired. If desired by the surgeon, the trigger may be partially depressed to move the cutting arm into contact with the bone prior to resecting it to ensure the desired bone area is properly targeted.

As described herein, one of the objects of the invention is to provide a powered surgical bone resector. Another object of the invention is to provide a bone resector that has a pneumatically powered cutting operation, with the pneumatic supply being alternatively from a sterile gas source such as a hand held canister or filtered air from a compressor system. Another object of the invention is provide a surgical bone resector that can do multiple resections while remaining within the surgical incision. Another object of the invention is to provide a surgical bone resector that automatically collects the bone specimens being resected. Another object of the invention is to provide a surgical bone resector that can automatically sort the bone specimens that are resected into any one of a multiple number of collecting bins. Another object of the invention is to provide a surgical bone resector that after resection automatically clears the bone specimen from the cutting arm. Another object of the invention is to provide a surgical bone resector that sorts the bone specimens into multiple disposable collecting bins. Another object of the invention is to provide a surgical bone resector which can position the cutting arm adjacent a selected bone tissue prior to selectively actuating a cutting operation. Another object of the invention is to provide a system of exchangeable and replaceable cutting arms and carriers, which detach easily from the handheld carrier. Another object of the invention is to provide cutting arms and carriers optimized for field of view, targeting, reaching and cutting in general or specific surgical procedures. Another object of the invention is to provide a powered surgical bone resector that is electronically controlled. Another object of the invention is to provide a surgical bone resector that is electronically controlled with no electrical wires or connections within proximity of the patient. Another object of the invention is to provide a surgical bone resector that has an electronic control that is programmable. Another object of the invention is to provide a method of resecting a bone with a powered bone resector. Another object of the invention is to provide a method of doing multiple resections with a bone resector while the resector remains within the surgical opening. Another object of the invention is to provide various methods for using the apparatus comprising the foregoing invention objects as well as those in the claims. The foregoing objects of the invention, and others as defined by the claims appended hereto, are achieved by the preferred embodiment of the invention disclosed herein. However, the preferred embodiment is provided not as a limitation of the invention but instead is to only be considered as illustrative thereof.

While several of the principal advantages and features of the invention have been discussed above, a fuller understanding of the invention may be attained by reading the following detailed description of the preferred embodiment and referring to the drawings included herein.

### BRIEF DESCRIPTION OF THE DRAWINGS

FIG. 1 is a perspective view of the Kerrison™ Rongeur bone resector of the prior art;

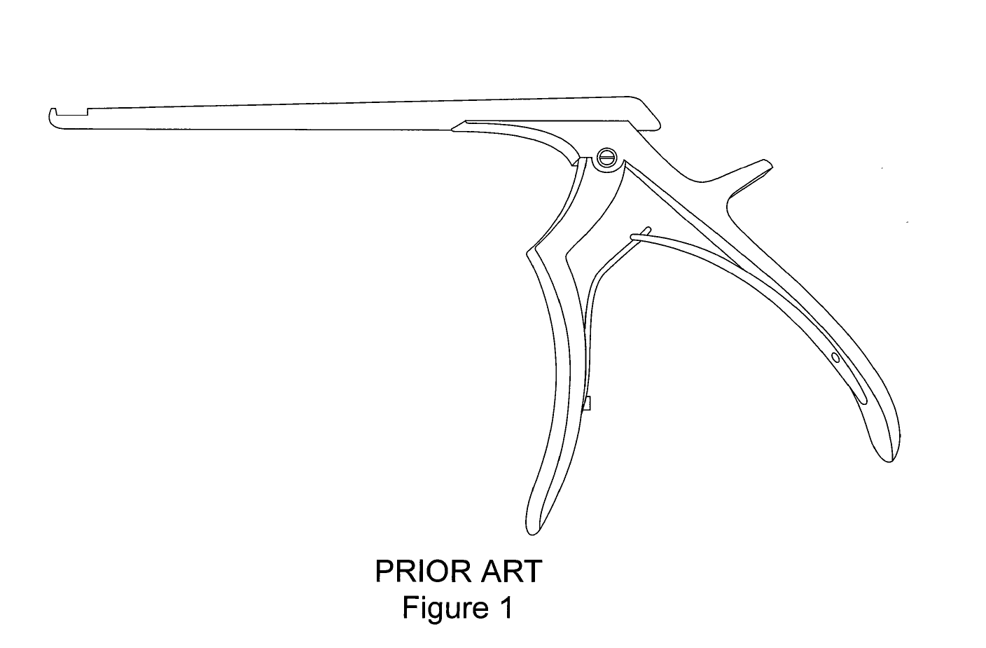

FIG. 2 is a perspective view of the pneumatically powered bone resector of the present invention; and

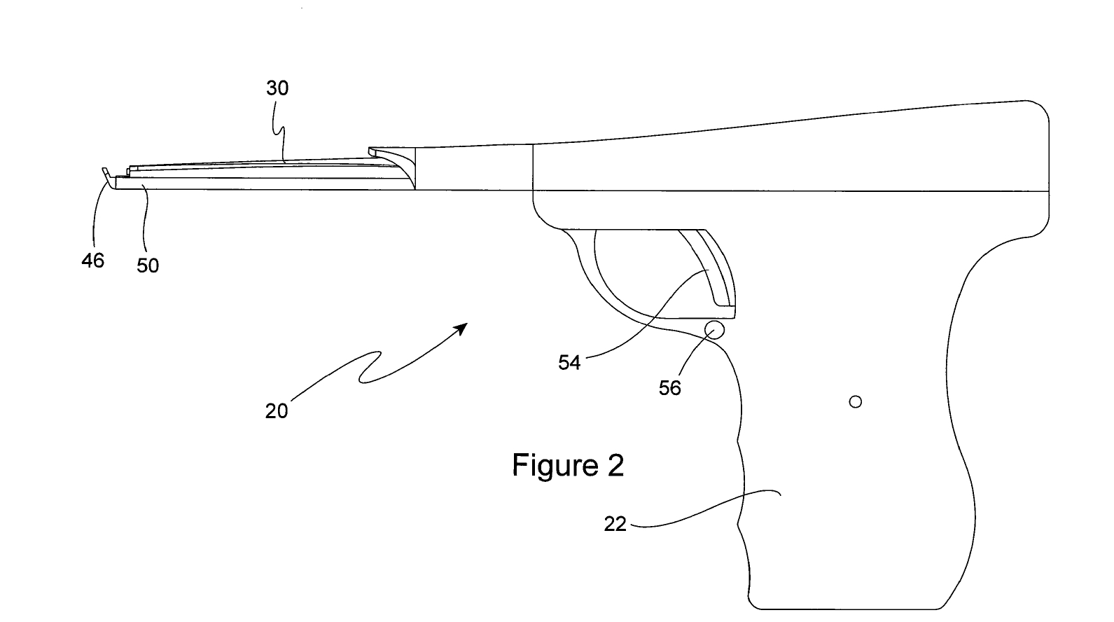

FIG. 3 is a cut away view of the interior assembly of the pneumatically powered bone resector of the present invention.

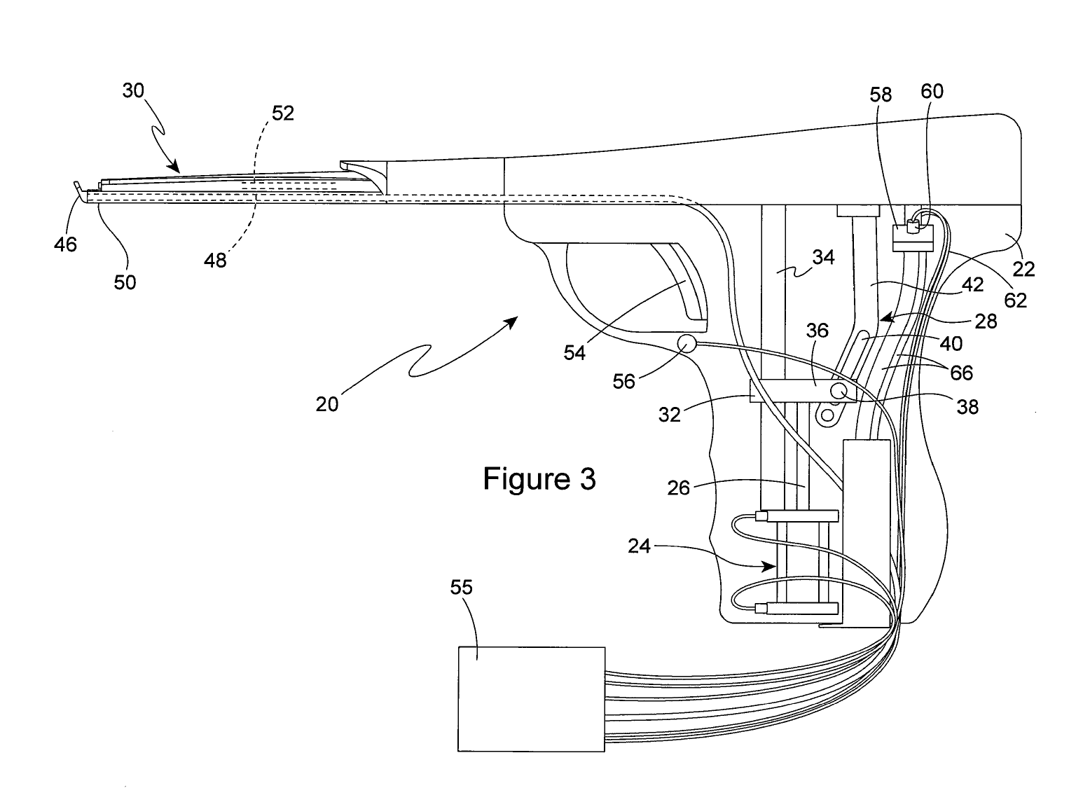

### DETAILED DESCRIPTION OF THE PREFERRED EMBODIMENT

The bone resector 20 of the present invention comprises a hand held carrier 22 preferably shaped in the form of a handgun for ease and familiarity of use by a surgeon. Inside the carrier 22 is mounted a dual action pneumatic cylinder 24 having its piston 26 connected to a mechanical linkage 28 for driving and retracting a cutting arm 30. The mechanical linkage 28 comprises a sliding yoke 32 attached to the piston 26 with a hole therethrough which mounts the sliding yoke 32 to a guide post 34 to guide it as the pneumatic cylinder is actuated. The yoke 32 has two yoke arms 36 which are bridged by a pin 38 extending through a slot 40 in a second guide arm 42 so that as the cylinder is actuated, the piston extends to drive the yoke along the first guide post and move the pin through the second guide arm, thereby pushing the second guide arm forward to drive it forwardly of the carrier 22. The cutting arm 30 is slidingly mounted to the top of the carrier 22 and is connected to the second guide arm 42 so that it is driven thereby. The carrier 22 has a stationary foot 46 extending upwardly at the end of the carrier 22 and against which the cutting arm 30 is driven to resect bone. The carrier 22 also has a tube, or pneumatic passage 48 along the extended forward nose 50 and through which pressurized air is blown, as is explained in greater detail below. The cutting arm 30 also has a pneumatic passage 52 in its interior, or is hollow, and which aligns with the pneumatic passage 48 as the cutting arm 30 is extended against the foot 46 so that resected bone may be blown out of the foot 46 and backwards through the cutting arm 30. A trigger 54 is positioned on the carrier 22 in the place of an ordinary handgun trigger, and when depressed sends a signal to the electronically controlled pneumatic power supply 55 to cause a bone resection to occur and then retract. Additionally, a switch 56 is arranged beneath the trigger 54 to allow a surgeon to select where the resected bone is collected. At the back end of the pneumatic passage 48 is connected a sorting bin 58 into which the resected bone is deposited after being blown backwards from the foot 46. The sorting bin 58 has a pair of ports 60 which are connected to pneumatic lines 62 and are arranged so that one of the other will be activated to blow resected bone deposited in the sorting bin into either one of two collection bins 64 through supply tubes 66. The collection bins 64 are preferably “locked” into the carrier 22 similarly as ammo clips are in a handgun, again for ease of operation and familiarity of use. The collection bins 64 preferably have on their tops compliant interlocking triangular flaps of rubber or other flexible material that allow passage of bone chips into the collection bins, but not out of the collection bins.

There are pneumatic lines extending preferably from the electronically controlled pneumatic power supply 55 to operate each side of the pneumatic cylinder 24, each of the two ports 60, and the bone resection pneumatic passages 48, 52. Electrical lines extend preferably from the trigger 54 and the switch 56 back as well to the electronically controlled pneumatic power supply 55. The power supply is controlled preferably by a programmable control, such as may be purchased from Gleason Research, Cambridge, Mass., under part no. GRHB-PC, and programmed as shown by the program in Attachment A. As would be apparent to one of ordinary skill in the art, other suitable programmable controls and options would be suitable as well. One such example would be to have the pneumatic control be under the control of a microcomputer, or the like. It is noted that with the preferred embodiment of the present invention the bone resector is electrically isolated from the patient as it is placed for bone resection within a surgical opening. The electrical wires and signals carried by to and from the trigger/switch are of low power and of no risk to any patient.

The pneumatic source may be any convenient source of pressurized air. The operating pressures found by the inventors to work in the preferred embodiment are from 80 psi to 100 psi. The timing for the turning on and off of the various (5 in the preferred embodiment) pneumatic lines is controlled from the running of the operating software as found in Attachment A. However, it would be expected that those or skill in the art, with the teachings of the present disclosure, would find it discernible without undue experimentation to determine operating pressures and timing for any convenient pneumatic power source.

The present invention preferably achieves bone resection by practicing the following method:

(1) A “cutting arm” of a desired size is selected from the set of cutting arms provided with the invention, and clipped into place on the carrier.

(2) The “collection bins” are inserted into the base of the carrier handle in the same way that an ammunition clip is inserted into the handle of a pistol.

(3) The device is plugged into a base unit comprising an electronically controlled pneumatic power supply containing control electronics and a compressed air purification system preferably using a high grade sterilizing filter to remove bacteriaphage from the pneumatic lines as may be purchased from the Shanghai Apureda Industry Co., Ltd. (not shown in detail).

(4) The “foot” at the cutting end of the device is placed beneath the bone to be resected.

(5) The “bin selection switch” is set to direct resected bone to one of the two “collection bins” through the sorting bin.

(6) If the surgeon chooses to do so, the trigger is depressed partially, allowing the cutting arm to contact the bone to be resected prior to cutting.

(7) The trigger is depressed fully, and the following set of actions follows:

(7a) A valve in the base unit sends pressure to the “pneumatic cylinder”

(7b) The pneumatic cylinder is actuated to drive the piston, which extends the yoke to ride up the “guide post”, pushing the “second guide arm”, which in turn pushes forward the cutting bar

(7c) The cutting arm slices through the bone, entrapping a bone chip between its forward tip and the foot

(7d) The control unit sends air through the base of the cutting arm to blow the bone chip away from the foot and backward through the handle

(7e) The bone chip is blown into the sorting bin, and then through the pre-selected supply tube to its associated collection bin, using the air powered sorter when needed

(7f) The control unit sends a signal to the cylinder to retract the piston, thereby allowing the mechanical linkage and cutting arm to retract to return to a home position to ready for another resection

(8) The surgeon repeats this process any number of times, without having to remove the device from the surgical entry, by depressing the trigger again each time

(9) When resection is complete, the “collection bins” are removed from the handle. Bone chips and tissue are removed from the bins, and the bin is either discarded or sterilized for reuse.

While the preferred embodiment has disclosed a preferred form and method of practicing the invention, there are changes, modifications, and alternatives that would be apparent to those of ordinary skill in the art and which are within the scope of the present invention. As such, the present invention should be considered as limited only by the scope of the claims appended hereto and their legal equivalents.

What is claimed is:

1. A bone resector having a powered cutting arm.

2. The bone resector of claim 1 further comprising a trigger controlling the power to said powered cutting arm, said trigger being operated to move the cutting arm and thereby resect a bone upon orienting the cutting arm to be in contact with said bone.

3. The bone resector of claim 2 further comprising a mechanical/pneumatic linkage connected to said cutting arm, said mechanical linkage being actuated by said trigger to thereby power the cutting arm.

4. The bone resector of claim 3 wherein said mechanical/pneumatic linkage comprises a pneumatic cylinder.

5. The bone resector of claim 4 further comprising an electronically controlled pneumatic power supply connected to said pneumatic cylinder, said trigger being connected to said electronically controlled power supply to controllably drive said cutting arm.

6. The bone resector of claim 5 wherein said cutting arm is hollow, and wherein said pneumatic power supply is connected to said cutting arm to controllably blow pressurized air through said hollow cutting arm and thereby clear it of resected bone.

7. The bone resector of claim 6 wherein pneumatic power supply blows pressurized air through said hollow cutting arm in response to said trigger being operated with an extended depression, longer than that required to operate the cutting arm.

8. The bone resector of claim 6 further comprising a sorting bin, said sorting bin being connected to said hollow cutting arm to receive resected bone blown therethrough.

9. The bone resector of claim 8 further comprising at least two collection bins in communication with said sorting bin, and wherein the pneumatic power supply is connected to said sorting bin to direct resected bone to one of said collection bins.

10. The bone resector of claim 9 further comprising a switch connected to said electronically controlled power supply, said switch being operable to designate a collection bin for receiving the resected bone.

11. A pneumatically powered bone resector having a moveable cutting arm and a stationary foot against which said cutting arm homes to cut bone positioned therebetween, said cutting arm being hollow for receiving bone resected thereby, a mechanical/pneumatic linkage connected to said cutting arm for driving said cutting arm into said foot, an electronically controlled pneumatic power supply for controllably powering said mechanical/pneumatic linkage upon operation of a trigger, said trigger being connected to said electronically controlled pneumatic power supply to permit operator initiated movement of said cutting arm to thereby resect said bone.

12. The bone resector of claim 11 further comprising a sorting bin in communication with said cutting arm to receive bone resected thereby, and at least two collection bins in communication with said sorting bin, said collection bin being in communication with said pneumatic power supply to thereby blow resected bone in a predetermined collection bin.

13. The bone resector of claim 12 wherein said mechanical/pneumatic linkage comprises a cylinder to drive the cutting arm into contact with said foot and to retract said cutting arm away from said foot to thereby reset the cutting arm for another bone resection.

14. The bone resector of claim 13 further comprising a switch connected to said electronically controlled pneumatic power supply for predetermining the collection bin into which resected bone is to be blown.

15. A method for repeatedly resecting bone with a powered bone resector having a powered, hollow cutting arm and stationary foot, said method comprising:
placing the bone desired to be resected between the end of the cutting arm and the foot,
actuating the bone resector to resect tissue from said bone,
transporting the bone tissue through the hollow cutting arm to a collection point, and
power retracting the cutting arm in preparation for the next bone resection.

16. The method of claim 15 further comprising repeating the bone resection by placing a new portion of the bone between the end of the cutting arm and the foot and then performing each of the recited processes.

17. The method of claim 16 wherein the powered bone resector includes a plurality of collection bins, and wherein transporting the bone tissue includes transporting the bone tissue to one of said collection bins.

18. The method of claim 17 further comprising designation of one of said collection bins for receiving resected bone tissue.

19. A method for clearing resected bone tissue from between a foot and a tip of a hollow cutting arm of a bone resector comprising transporting said bone tissue through the cutting arm to a collection point.

20. The method of claim 19 wherein transporting includes blowing said issue through the length of said cutting arm.
